(19) United States
(12) Patent Application Publication
(10) Pub. No.: US 2003/0201224 A1
(43) Pub. Date: Oct. 30, 2003

(54) Microbial consortium for the biodegradation of dithiocarbamates

(76) Inventors: James E. Gannon, Bonner, MT (US); Mike Standish, Rock Spring, GA (US)

Correspondence Address: Thomas F. Roland, NATIONAL STARCH AND CHEMICAL COMPANY, P.O. Box 6500, Bridgewater, NJ, 08807-0500, US

(21) Appl. No.: 10/131,901

(22) Filed: Apr. 24, 2002

(51) Int. Cl. C12N 1/20; E03B 11/00

(52) U.S. Cl. 210/601; 435/252.4

(57) ABSTRACT

The invention relates to a method for biodegrading dithiocarbamates or related compounds which are present in a contaminated environment. The method involves contacting the contaminated environment with a microbial consortium comprised of methylotrophic bacteria such as the genera of bacteria: Alcaligenes, Pseudomonas, and Hypomicrobium, and maintaining the microbial consortium in contact with the contaminated environment for a time that is sufficient for the microbial consortium to degrade the dithiocarbamates or related compounds. Other bacterium, such as Thiobacillus may optionally be present as part of the consortium.

### FIELD OF THE INVENTION

This invention relates to a microbial consortium useful for biodegrading dithiocarbamates.

### BACKGROUND OF THE INVENTION

Dithiocarbamates are used in a variety of water treatment applications as metal precipitating agents. Dithiocarbamates provide a cost effective means of removing heavy metals from metal processing wastewater. Dithiocarbamates, however, pose a toxicity problem. In particular, dithiocarbamates have been shown to be inherently toxic to fish and other wildlife. In addition, dithiocarbamates may with time and under certain conditions autocatalytically hydrolyse to carbon disulfide which also poses a toxicity problem.

Mounting public concern and increasing environmental legislation have provided the impetus for a safe, effective means to remediate dithiocarbamates contaminated environments. Past methods of disposing of wastewater or soil containing dithiocarbamates have included dumping at specified land-fill areas, isolation in suitable, reinforced containers, land based deep-welling, dumping in deep water at sea and incineration. All of these methods carry some potential for harm to the environment. For example, incineration creates a problem of air pollution and disposal on land risks the possibility that toxic substances will leach into locations where they may threaten aquatic life forms, animals or humans. A more desirable disposal method might incorporate a chemical, enzymatic, or biological degradative process.

The metabolic reduction of dithiocarbamates is reported. J. Kaslander, “Metabolic Fate of Dithiocarbamates”, dissertation, University of Utrecht (1966) describes the assimilation of sodium dimethyl dithiocarbamate (SDM) into the α-amino butyrate derivative. V. S. Brozel, B. Pietersen, and T. E. Clote, “Resistance of Bacterial Cultures to Non-Oxidising Biocides”, Wat. Sci. Tech. 31, 169-175 (1995) describes bacterial resistance to sodium dimethyl dithiocarbamate but not degradation of sodium dimethyl dithiocarbamate Non-biological oxidation of SDM can result in the formation of tetramethyl thiuram disulfide. Microbial degradation of tetramethyl thiuram disulfide has been reported (C.K. Shirkot and K.G. Gupta. Accelerated tetramethyl thiuram disulfide degradation in soil by inoculation with a TMTD-utilizing bacteria”, Bull. Environ. Contam. Toxicol. (1985) 35:354-361; C.K. Shirkot, P. Shirkot, and K.G. Gupta. “Isolation from soil and growth characteristics of the tetramethyl thiuram disulfide (TMTD) degrading strain of pseudomonas aeruginosa. (1994) J. Environ. Sci Health, A29(3) 605-614)). These authors, however, did not demonstrate the bacterial degradation of SDM as breakdown product of tetramethyl thiuram disulfide above the levels expected in non-biological controls. One additional paper also describes the microbial degradation of TMTD (K. Maeda and T. Tonomura “Microbial degradation of tetramethyl thiuram disulfide”, (1968), Kenkyau haokoku (Kaogyao Gijutsuin Biseibutsu Kaogyao Gijutsu Kenkyaujo (Japan) 33(1): 1-8) and identifies the degradation products as dithiocarbamate, dimethylamine, formaldehyde, elementary sulfur, and methionine.

There remains a need for an effective degradation process for dithiocarbamates and related compounds that will degrade those compounds completely and is effective in both the in vitro and in situ remediation of contaminated environments including both soil and water systems.

### SUMMARY OF THE INVENTION

Accordingly, it is an object of the invention to provide a microbial consortium capable of biodegrading dithiocarbamates.

It is another object of the invention to provide a method for biodegrading dithiocarbamates in which a microbial consortium is added to contaminated soil or water for the purpose of biodegrading dithiocarbamates present as contaminants in the soil or water.

It is also an object of the invention to provide a method for biodegrading dithiocarbamates to substances which are environmentally safe.

With regard to the foregoing and other objects, the present invention provides a method for biodegrading dithiocarbamates or related compounds which are present in a contaminated environment, said method comprising contacting the contaminated environment with a microbial consortium comprising methylotrophic bacteria including a number of bacteria such as Alcaligenes, Pseudomonas, Hypomicrobium, and other methylotrophs, and optionally other bacterium such as Thiobacillus, maintaining the microbial consortium in contact with the contaminated environment for a time that is sufficient for the microbial consortium to degrade the dithiocarbamates or related compounds.

For its use in the course of decontamination of contaminated soils and waters, the consortium is applied to environments having a pH-value of between 5.0 and 8.5 and in a temperature range of from 5° C. to 42° C. The consortium is applied in a quantity that results in a final soil concentration of greater than 10⁶ cells per gram of contaminated soil. In water, the consortium is applied at a level resulting in a concentration of greater than 10⁶ cells/ml. The amount of consortium containing material applied would depend on the density of cell mass in the consortium preparation, and this can be adjusted by dilution or concentration techniques. Likewise, the consortium may be applied in a dried mass absorbed to carrier particles such as shredded waste agricultural stock.

The process for its use includes basically the one-time or several-time spraying (dissolved in water) or spreading of the mixture (dried) on to the contaminated mass and afterwards the optimization of the milieu by aeration or nutrient addition. For waste stream purification, the process includes developing the consortium on a solid matrix such as gravel or other appropriate fill material and optimizing the flow of the waste stream through the bed to achieve degradation.

According to another aspect the invention provides a microbial consortium comprised of multiple genera of bacteria consisting of Pseudomonas and Hyphomicrobium species with secondary amounts of, Alcaligenes and other species of facultative methylotrophs. The consortium could optionally contain other bacteria, such as Thiobacillus. In one preferred embodiment, the consortium contains Thiobacillus bacteria. The microbial consortium has been isolated from a contaminated environment and is capable of biodegrading dithiocarbamates or related compounds.

The microbial consortium when applied to a contaminated environment biodegrades dithiocarbamates and related compounds to intermediates such as sulfide and dimethylamine which are then oxidized to carbon dioxide, water, ammonia, and sulfate. The microbial consortium is effectively applied to soil, water, treatment ponds, treatment ditches, waste disposal sites, and waste streams which are contaminated with dithiocarbamates or related compounds.

### BRIEF DESCRIPTION OF THE DRAWING

FIG. 1 shows the percent degradation of dithiocarbamate by the consortium over time.

### DETAILED DESCRIPTION OF THE INVENTION

The invention relates to a novel microbial consortium capable of biodegrading dithiocarbamates and related compounds. The dithiocarbamates or related compounds are present as contaminates in the environment. As used herein, “contaminated environment” or “contaminated environments” means any environment contaminated with dithiocarbamates or related compounds. Typical contaminated environments may include, but are not limited to, soil, water, treatment ponds, treatment ditches, manufacturing facilities, waste disposal sites, and waste streams.

The dithiocarbamates may be in the form of a liquid, solid or combination thereof. The dithiocarbamates include, but are not limited to sodium diethyl dithiocarbamate, sodium dimethyl dithiocarbamates, sodium dipropyl dithiocarbamates, and sodium dibutyl dithiocarbamates.

Isolation of and Composition of the Consortium

As used herein, “microbial consortium” refers to any collection of microorganisms which are capable of biodegrading dithiocarbamates.

The microbial consortium of the present invention was isolated from a waste treatment facility of an industrial site and selected by the following method. A sample of soil was inoculated into minimal medium (buffered mineral salts) supplemented with 100 ppm of sodium dimethyl dithiocarbamate, 500 ppm of dimethyl amine, vitamins and yeast extract, and incubated for 7 days at 25° C. The culture was further subcultured into minimal medium supplemented with 250 ppm of sodium dimethyl dithiocarbamate, 250 ppm of dimethyl amine, vitamins and yeast extract, and incubated and additional 7 days at 25° C. The culture was further subcultured into minimal medium supplemented with 500 ppm of sodium dimethyl dithiocarbamate, 100 ppm of dimethyl amine, vitamins and yeast, and incubated until visible cellular turbidity as observed at 25° C. The above process was repeated an additional two times until the final enrichment contained sodium dimethyl dithiocarbamate at 1000 ppm and no dimethylamine.

The concentration of sodium dimethyl dithiocarbamate was monitored during the isolation procedure by a calorimetric assay which involved forming a copper-sodium dimethyl dithiocarbamate complex and measuring color formation. The assay involved combining 1 ml of water, 1 ml of copper-acetate (0.103 g/100 ml) and 2 ml of sample. Color development was determined by measuring absorbance at 430 nm. A reduction in absorbance indicated removal of dithiocarbamate. One isolated group of bacteria, herein called the microbial consortium, proved to rapidly reduce the concentration of dithiocarbamate and has maintained this ability through a number of successive transfers indicating the consortium has the ability to degrade dithiocarbamate and that this is a stable trait.

Analysis of the members of the consortium has been completed by a number of approaches. Several methods indicated that there were at least four and as many as 7 different bacterial types present in the consortium including the species Alcaligenes, Pseudomonas, and Hypomicrobium. Only the Alcaligenes spp. and the Pseudomonas spp. are culturable using conventional culturing techniques. The presence of the putative Hyphomicrobium spp. was determined by visual microscopic (Hyphomicrobium morphology) and molecular biology techniques.

Since it is difficult, at best, to accurately characterize a mixed consortium of microorganisms, several different approaches had been utilized. The difficulty in characterizing environmental microorganisms is well know in the field and results from two general observations, 1) many bacteria cannot be cultured by themselves in the laboratory although they may be repeatedly grown in a complex mixture (consortium), and 2) describing a particular bacterium as a precise “species” is not always possible and there may not be a absolute designation. In bacteria, the concept of species is much debated (see J.T. Staley (1999), ASM News vol. 65(10) 681-687). Therefore the following descriptions of the members of the consortium must consider this.

Standard bacteriological plating techniques on different growth media have revealed that the majority of culturable bacteria in the consortium could be assigned to the genus Pseudomonas/Hypomicrobium, and Alcaligenes. Since plating on bacteriological medium generally only reflects a fraction of an environmental population, the consortium has also been analyzed using molecular techniques. Purified DNA from the consortium was subjected to the polymerase chain reaction using primers specific for the 16SrRNA genes. The amplified genes were then separated and analyzed using density gradient gel electrophoresis (DGGE) as described by Muyzer et al., 1993 (Appl. Environ. Microbiol. 59, 695-700). Each band on the resulting gel indicates a unique microorganism. DGGE results suggest that there are a minimum of 4 and a maximum of 7 unique bacteria in the consortium. Most of these appear to be Pseudomonas, Hyphomicrobium, and Alcaligenes. Phase contrast microscopy has also confirmed that some members have a characteristic Hyphomicrobium morphology. Finally, many types of bacteria have unique signature fatty acids. The total lipid from the consortium were extracted, separated by polarity using column chromatography and the fatty acid fraction purified, derivatized and characterized by gas-liquid chromatography. This analysis confirmed the presence of methanol-utilizing Pseudomonas and Hypomicrobium spp. The results are consistent of the Type B methylotrophs of Urakami and Komagata (J. Gen Appl. Microbiol., 25: 343-360 (1979) which include Pseudomonas, Hyphomicrobium, Methylobacillus, Acetobacter, and Xanthomonas and others.

In summary, the consortium is a mixture of mostly methylotrophic bacteria dominated by Pseudomonas, Alcaligenes, and Hyphomicrobium. Other methylotrophs and Thiobacillus species may be present in minor amounts. No single culturable member of the microbial consortium has demonstrated the ability to biodegrade dithiocarbamates when inoculated into medium containing sodium dimethyldithiocarbamate as sole carbon source. It appears that it is necessary for a microbial consortium of at least several type B methylotropic microorganisms together for complete dithiocarbamate degradation to occur.

This consortium has been deposited with the American type Culture collection under the terms of the Budapest Treaty and has been assigned the assession number as ATCC XXXXX.

For its use in the course of decontamination of soils and waters, the consortium is best prepared in a synthetic medium (comprised potassium phosphate buffer 0.02M (pH 6-8), ammonium nitrate (0.5 g/l), potassium chloride (0.25 g/l), magnesium sulfate heptahydrate (0.25 g/L) and between 500 and 2000 ppm SDM and between 100 and 1000 ppm dimethylamine. It is best grown between 20-35° C., the cultivation time between 4 to 7 days depending on the requirements of the density of the bacterial suspension. It may then be lyophilized (freeze dried) or prepared as a concentrated slurry by removal of water.

The preparation may be used by spraying or spreading on to contaminated soil or by inoculation into contaminated water. The method also allows containment of the contaminated soil or water in a bioreactor (tank) followed by inoculation with the consortium. The method further allows establishment of an attached consortium on a solid substrate such as gravel and thereby establishing a flow through reactor where the flow rate is regulated to maximize degradation of the dithiocarbamate. In all cases the microbial mixture is added in a quantity in which the final cell density is greater than about 1.0×10⁶ consortium members per gram of soil or per ml of water. Lesser amounts may be initially applied, and the number of cells allowed to increase though cell growth, however the rate of degradation will be significantly slower. A preferred level of consortium members is about 10⁷ per gram of soil or per ml of water. The pH value of the treated medium preferably is kept between 5.0 and 8.5 and the degradation process is continued under aerobic conditions.

It has been found that preparations of the consortium generally contain about 10⁸ cells/ml. Thus to treat 100 liters of water, with a desired treatment level of 10⁷ cells/ml, one would add 10 liters of the consortium having 10⁸ cells/ml to 90 liters of water. If the concentration of the consortium is higher than 10⁸/ml, then a lesser treatment could be used. For a faster rate of degradation, a greater volume of consortium could be used. It has been found that generally about 10 to 20 percent (volume/volume) of the consortium to aqueous system is an appropriate treatment level.

An example of a treatment of a soil system would be the treatment of a contaminated soil of one square meter contaminated to a depth of 6 inches. This would represent approximately 152,400 cm³. To obtain a treatment level of 10⁷ cells per gram of soil, and assuming a dried consortium cell concentration of about 10¹¹ cell/g, about 22 grams of dried consortium would be required for this square meter.

The result of the application of the inventive mixture of natural microorganisms is a degradation of the dithiocarbamate structure in the environment to mostly carbon dioxide and cell mass.

The advantages of the consortium and its uses are to be seen in the fact that the created metabolites are not toxic. The only potential toxic byproduct of metabolism is carbon disulfide and/or hydrogen sulfide, however, degradation of carbon disulfide to carbon dioxide is the putative role of the Thiobacillus spp. in the consortium as is known (S. L Jordan, A. J. Kraczkiewicz-Dowjat, D. P. Kelly, and A. P. Wood, “Novel eubacterium able to grow on carbon disulfide (1995), Arch. Microbiol. 163: 131-137). Also a number of microorganisms (sulfur oxidizing) can oxidize hydrogen sulfide. While the exact mechanism of degradation is not known, it is very likely that the first step in the degradation of the dithiocarbamates is due to the oxidation of one of the sulfur groups by a consortium containing monooxygenase. When this occurs, at neutral pH, one of the sulfurs is released as a sulfide and the remaining part of the dithiocarbamate structure becomes cell associated and is eventually assimilated into cell mass. The ability to do this is specific to this consortium in that a number of other bacteria and enrichments have been evaluated and no enzyme mediated oxidations are observed.

A further advantage is that the number of beneficial soil microorganisms in the soil is increased improving the structure of the soil and converting the carbon, nitrogen, and sulfur entrained as dithiocarbamate back to inorganic elements for use by plants and soil microorganisms. In view of the fact that the organisms used are isolated from the natural environment and that they are not genetically engineered, there is no danger for a negative influence on the biosphere whatsoever.

The following nonlimiting examples illustrate further aspects of the invention.

### EXAMPLES

The following materials and methods were used in the Examples:

(1) Sodium dimethyl dithiocarbamate (SDM) or dimethyl dithiocarbamate acid, sodium salt hydrate was obtained from Fluka Chemical as a 40% solution in water.

(2) Sodium Dipropyl dithiocarbamates and Sodium Dibutyl dithiocarbamates were synthesized at Alco Chemical Corporation. Spectrophotometric determinations were performed using a Beckman DU-6 spectrophotometer.

### Example 1

Biodegradation of SDM in aqueous systems.

A 1000 ml bioreactor containing the synthetic medium (described above) has been inoculated with the consortium density of approximately 1.1×10⁷ ml. The initial pH value was 7.2 and the temperature varied between 20-27° C. The consortium density was measured by direct epifluorescent microscopic counts using the DNA specific dye 4,6-diamidino-2-phenylindole. SDM concentration was measured spectrophometrically by complexing with cupric acetate and measuring the absorbance at 430 nm.

TABLE I

| | Cell Count × | | Consortium | Control (w/o |
|---|---|---|---|---|
| | 10⁻⁵ | | added | consortium |
| Time (h) | Average | STD | SDM (mg/L) | SDM (mg/ml) |
| 0 | 111 | 6.4 | 988 | 959 |
| 48 | 203 | 11.4 | 205 | 923 |
| 80 | 541 | 51 | 98 | 871 |
| 120 | 836 | 69 | 21 | 825 |
| 168 | 1071 | 103 | <20 | 812 |
| 312 | 933 | 85 | <20 | 794 |

### Example 2

### Degradation of an Industrial Waste Effluent

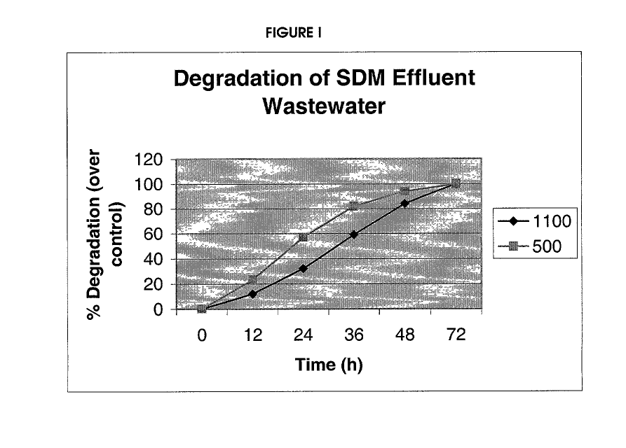

To further demonstrate the degradation of dithiocarbamate by the consortium, an actual waste sample, waste wash-water, from an industrial production plant was evaluated. The sample contained about 1100 ppm of SDM. The sample, and a dilution of it at 500 ppm and pH 7.8, was inoculated with a 20% (VN) amount of a 72 h consortium. A control without inoculation was included. The results shown in FIG. 1 show that the consortium degraded almost 100% of the SDM within a 72 hour period.

### Example 3

Biodegradation of Sodium Dimethyidithiocarbamate by Microbial Consortium in Soils

Two one-kilogram soil samples were contaminated with from 100 to 5000 mg of SDM. One soil environment was inoculated with approximately 1.2×10⁷ consortium members/g soil while the other remained uninoculated. Nitrogen and phosphorus was added to both systems. The initial soil pH was 6.8 and the temperature varied from 18° C. to 27° C. Water was added to maintain the dry weight at about 30%. Each soil system was mixed weekly to provide aeration. At the indicated periods, soil aliquots were extracted with a methanol:chloroform:water (1:1:0.9) and the SDM concentration determined as indicated above.

TABLE II
PERCENT degradation of SDM

| Time (d) | 100 | 500 | 1000 | 2000 | 5000 |
|---|---|---|---|---|---|
| 1 | 1.6 | 6.2 | 4.8 | 3.9 | 5.2 |
| 3 | 18.3 | 22.6 | 16.3 | 12.4 | 3.1 |
| 7 | 50.2 | 63 | 39.7 | 26.7 | 2.3 |
| 10 | 81 | 88 | 48 | 32.1 | 1.8 |
| 14 | 87.3 | 92 | 72 | 47.6 | 3.6 |
| 20 | 91 | 91 | 76 | 54 | 1.8 |
| 28 | 92.4 | 89 | 81 | 58 | 3.2 |

% degradation was determined by comparison with uninoculated controls

### Example 4

The following example demonstrates other N-alkyl dithiocarbamates that can also be degraded by the consortium.

A 1000 ml bioreactor containing the synthetic medium (described above) has been inoculated with the consortium density of approximately 1.1×10⁷ ml. The initial pH value was 7.2 and the temperature varied between 20-27° C. Dithiocarbamate concentration was measured spectrophometrically by complexing with cupric acetate and measuring the absorbance at 430 nm. The results show that the consortium is capable of degrading a variety of N-alkyl dithiocarbamates to varying degrees.

| | % Diethyl | % DiPropyl | % Dibutyl | % Dimethyl |
|---|---|---|---|---|
| Time (h) | Dithiocarbamate* | Dithiocarbamate | Dithiocarbamate | Dithiocarbamate |
| 0 | 3.28 | 3.99 | 4.44 | 3.02 |
| 0.5 | 2.80 | 3.87 | 4.70 | 8.1 |
| 1.5 | 8.78 | 4.21 | 4.10 | 9.5 |
| 4 | 8.18 | 3.56 | 3.81 | 12.3 |
| 5 | 13.29 | 3.49 | 3.98 | 12.1 |
| 7 | 15.01 | 4.37 | 4.12 | 27.1 |
| 27 | 18.00 | 7.82 | 6.11 | 61.4 |
| 51 | 21.00 | 9.43 | 6.66 | 77.8 |
| 73 | 23.00 | 11.24 | 7.93 | 88.7 |
| 96 | 25.07 | 14.56 | 9.46 | 97.5 |

*% degradation is calculated as percent loss over the abiotic Control

What is claimed is:

1. A microbial consortium comprised of methylotrophs comprising the bacterial genera Alcaligenes, Pseudomonas, and Hypomicrobium, said microbial consortium being capable of biodegrading dithiocarbamates or related compounds.

2. The microbial consortium of claim one wherein said consortium comprises 40 to 70 percent Pseudomonas, and Hypomicrobium bacterium and from 20 to 40 percent Alcaligenes bacterium.

3. The microbial consortium of claim 1 further comprising Thiobacillus bacteria.

4. A method for biodegrading dithiocarbamates or related compounds which are present in a contaminated environment, said method comprising contacting the contaminated environment with a microbial consortium comprised of methylotrophic bacteria comprising Alcaligenes, Pseudomonas, and Hypomicrobium bacterium and maintaining the microbial consortium in contact with he contaminated environment for a time that is effective for the microbial consortium to degrade the dithiocarbamates or related compounds.

5. The method of claim 4 wherein the concentration of the microbial consortium in contact with said contaminated environment is greater than 10⁶ cells per gram of contaminated soil, or 10⁶ cells per milliliter of contaminated water.

6. The method of claim 5 wherein the concentration of the microbial consortium in contact with said contaminated environment is greater than 10⁷ cells per gram of contaminated soil, or 10⁷ cells per milliliter of contaminated water.

7. The method according to claim 4 wherein the microbial consortium is produced by culturing a naturally-occurring population of microorganisms in a medium comprising dithiocarbamates.

8. The method according to claim 4 further comprising the step of adding to the contaminated environment a member selected from the group consisting of a nutritional source of nitrogen and a nutritional source of phosphorous for the microbial consortium.

9. The method according to claim 4 further comprising the step of adding water to the contaminated environment.

10. The method according to claim 4 wherein the contaminated environment is a member of the group consisting of a water and an aqueous slurry of soil or other particulate matter.

11. The method according to claim 4 wherein the contaminated environment is maintained at a pH within a range of 5.0 to 8.5.

12. The method according to claim 4 wherein the contaminated environment is maintained at a temperature within a range of 5° C. to about 42° C.

13. The method according to claim 4 further comprising the step of sequestering the contaminated environment in a vessel.
